(19) United States
(12) Patent Application Publication
(10) Pub. No.: US 2003/0138951 A1
(43) Pub. Date: Jul. 24, 2003

(54) Conversion of liver stem and progenitor cells to pancreatic functional cells

(76) Inventor: Li Yin, Gainesville, FL (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 10/273,746

(22) Filed: Oct. 18, 2002

Related U.S. Application Data
(60) Provisional application No. 60/337,446, filed on Oct. 18, 2001.

(51) Int. Cl. C12N 5/08

(52) U.S. Cl. 435/370

(57) ABSTRACT

The subject invention a method for converting liver stem/progenitor cells to a pancreatic functional cell by transfecting said liver cells with a pancreatic development gene and/or by culturing with pancreatic differentiation factors. The resulting cells produce and secrete insulin protein in response to glucose stimulation.

### RELATEDNESS OF THE APPLICATION

The subject application claims the benefit of priority from U.S. Ser. No. 60/337,446, filed Oct. 18, 2001.

### BACKGROUND

Cell Transplantation as a Cure or Treatment for Diabetes

Type I diabetes is a chronic metabolic disease caused by selective autoimmune destruction of insulin-producing islet β-cells. Clinical management of diabetes costs ˜$100 billion annually in this country. The insulin insufficiency and hyperglycemia of type I diabetes, in the long run, lead to serious secondary complications. Regular insulin replacement therapy that is being used to control daily glucose fluctuations, however, does not maintain glucose levels near-normal range at all times to prevent/reduce clinical complications (The DCCT Research Group (1991) N. Eng. J. Med. 329:977).

To cure or treat type I diabetes (both in terms of achieving insulin independence and reducing the incidence of secondary complications), it is essential to restore islet β-cells in the patients either as whole pancreas or islet transplantation. Only about 3,000 cadaver pancreata become available in the US each year while ˜35,000 new cases of type I diabetes are diagnosed during the same period of time (Hering, G. J. et al. (1999) Graft 2:12-27). Thus, there is an urgent need to develop alternate sources of functional cells of pancreatic lineage, including islets and/or insulin-producing cells. The only conceptual option available to circumvent the severe shortage of pancreatic tissue for transplantation is to develop functional cells of pancreatic lineage (e.g., islets or insulin-producing cells) in vitro from stem cells.

One source of transplantable islets is pancreas-derived islet producing stem cells (IPSCs) (Ramiya, V. K. et al. (2000) Nature Med. 6(3):278-282; and PCT/US00/26469, filed Sep. 27, 2000). However, additional/alternative methods of generating pancreatic lineage cells should be investigated to increase the chances of success in attempts to cure or treat type I diabetes. Liver stem/progenitor cells offer a feasible source for conversion into pancreatic lineage cells. There are many advantages of using liver stem cells: a) liver has the immense potential to regenerate following partial hepactectomy (for instance, the mass and function of the partially hepatectomized liver can be totally restored in about a week, even if ⅔ of liver is resected (Higgins, G. F. et al. (1931) Arc. Pathol. 12:186-202; Grisham, J. W. (1962) Cancer Res. 22:842-849; and Bucher, N. (1963) Int. Rev. Cytol. 15:245-300)), and therefore, liver provides a more easily accessible source of stem cells for autologous transplantation; and b) the surface phenotype of liver stem cells have already been established and hence it is easier to purify them from the organ (see Table 1). Also liver stem cells share surface hematopoietic stem cell markers like CD34, Thy1.1, stem cell factor(SCF)/c-kit, Flt-3 ligand/flt-3 (Yin, L. et al. (2001) Proc. Am. Assoc. Canc. Res. 42:354; Yin, L. et al. (2001) FASEB J. Late-Breaking Abstracts:49 (LB267); Fujio, K. et al. (1996) Exp. Cell Res. 224:243-50; Blakolmer, K. et al. (1995) Hepatology 21(6):1510-16; Omori, N. et al. (1997) Hepatology 26(3):720-27; Omori, M. et al. (1997) Am. J. Pathol. 150(4):1179-87; Lemmer, E. R. et al. (1998) J. Hepatol. 29:450-454; Petersen, B. E. et al. (1998) Hepatology 27(2):433-445; and Baumann, U. et al. (1999) Hepatology 30(1):112-117), which can be used for cell sorting along with other known liver stem cell markers.

TABLE 1
Markers of development, differentiation and cell
lineage specification of liver epithelial cells
(Adapted from Grisham et al. (1991) in Stem Cells, C. S. Potter (ed.),
Academic Press, San Diego, CA, pp. 233-82, with modifications).

| | | | | Bile |
|---|---|---|---|---|
| Markers | Hepatoblasts | Oval cells | Hepatocytes | duct cells |
| CK19 | ± | ± | = | ± |
| CK14 | ± | ± | = | = |
| Albumin | ± | +/− | ± | = |
| AFP | ± | ± | = | = |
| GGT | ± | ± | = | ± |
| OV6 | ± | ± | = | ± |
| OV1 | ± | ± | = | ± |
| BD1 | = | = | = | ± |
| HES6 | = | = | ± | = |
| OC.2 | ± | ± | = | ± |
| OC.3 | ± | ± | = | ± |
| OC.10 | ± | ± | = | ± |
| H.1 | = | = | ± | = |
| H.4 | = | = | ± | = |

The following sections describe the current status of liver and pancreatic stem cells, their relationship during embryonic development and transdifferentiation within these organs.

Development of Liver and Liver Stem Cells

In the embryo, liver buds from epithelial cells of the ventral foregut in the region that is in contact with the precardiac mesoderm, at approximately 8.5 to 9 days of development in the mouse. The cells of this region proliferate to form the liver diverticulum. At about 9.5 days of gestation, cells of the liver diverticulum begin to migrate into the surrounding septum transversum. At this stage, the cells are designated as hepatoblasts, to indicate that these cells have been determined along the hepatic epithelial cell lineage. The hepatoblast has bipotential capability, and gives rise to both hepatocytes and bile duct cells (Houssaint, E. (1980) Cell Differ. 9:269-279). In general, when the liver is injured, the mature hepatocytes proliferate to restore the mass and function of the liver, and the liver stem cells are not involved (Kelly, D. E. et al. (1984) in Bailey's Textbook of Microscopic Anatomy, 18^{th} ed., Williams and Wilkins, Baltimore, pp. 590-616). However, when the injury is too severe and/or the proliferation of hepatocytes is inhibited by chemicals such as 2-N-acetylaminofluorene (2AAF) and phenobarbital, the liver stem cell compartment is activated. Liver stem cells in the adult liver have been extensively studied mainly in the animal liver injury models, such as 2AAF/partial hepatectomy (PH) (Golding, M. et al. (1995) Hepatology 22(4): 1243-1253), 2AAF/allyl alcohol (AA) and phenobarbital/cocaine leading to periportal liver injury (Yavokovsky, L. et al. (1995) Hepatology 21(6):1702-12; Petersen, B. et al. (1998) Hepatology 27(4):1030-1038; Yin, L. et al. (1999) J. Hepatology 31:497-507; and Rosenberg, D. et al. (2000) Hepatology 31(4):948-955), and 2AAF/CCl₄ inducing pericentral liver injury (Petersen et al. (1998)). Irrespective of the injury site, the oval shaped liver stem cells always originate in the portal area of canals of Hering (Wilson, J. et al. (1958) J. Pathol. Bacteriol. 76:441-449). These liver progenitor cells in adult liver can differentiate into both hepatocytes and bile duct cells (Stenberg, P. et al. (1991) Carcinogenesis 12:225-231; and Dabeva, J. et al. (1993) Am. J. Pathology 143:1606-1620). Most recently, several lines of evidence from both animals and humans strongly suggest that hematopoietic stem cells are the extrahepatic source of liver stem cells (Petersen, B. et al. (1999) Science 284:1168-70; Theise, N. et al. (2000) Hepatology 31(1):235-40; Theise, N. et al. (2000) Hepatology 32(1):11-16; and Alison, M. et al. (2000) Nature 406:257). Epithelial cell lines with stem-like properties have been established from mouse liver diverticulum (Rogler, L. (1997) Am. J. Pathol. 150(2):591-602), injured rat liver (Yin, L. et al. (2001A) PAACR 42:354; Yin, L. et al. (2001B) FASEB J. Late-Breaking Abstracts:49 (LB267); Yin, L. et al. (2002) Hepatology 35(2):315-324), and normal rat (Tso, M -S. et al. (1984) Exp. Cell. Res. 154:38-52; and Tso, M -S. (1988) Lab. Invest. 58:636-642), porcine (Kano, J. et al. (2000) Am. J. Pathol. 156(6):2033-2043), and human liver (Crosby, H. et al. (2001) Gastroenterology 120(2):534-544). These cells can be induced to differentiate into hepatocytes and/or bile duct cells in vitro (Rogler, L. (1977); Yin, L. et al. (2001A); Yin, L. et al. (2001B); Yin, L. et al. (2002); Crosby, H. et al. (2001); and Coleman, W. et al. (1993) Am. J. Pathol. 142:1373-82) and in vivo upon transplantation (Coleman, W. et al. (1993); and Grisham, J. et al. (1993) Proc. Soc. Exp. Biol. Med. 204:270-79).

The signaling molecules that elicit embryonic induction of the liver from the mammalian gut endoderm are not fully understood. Fibroblast growth factors (FGFs) 1, 2, and 8 expressed in the cardiac mesoderm are reported to be essential for the initial hepatogenesis (Jung, J. et al. (1999) Science 284:1998-2003). Oncostatin M (OSM), an interleukin-6 family cytokine, in combination with glucocorticoid, induces maturation of hepatocytes in embryonic liver, which in turn terminate embryonic hematopoiesis. Livers from mice deficient for gp130, an OSM receptor subunit, display defects in maturation of hepatocytes (Kamiya, A. et al. (1999) EMBO J. 18(8):2127-36; and Kinoshita, T. et al. (1999) PNAS 96:7265-70). Differentiated hepatocytes are characterized by the expression of a unique combination of liver-enriched (but not liver-unique) transcription factors of HNF1, HNF3, HNF4, and C/EBP families (Johnson, P. (1990) Cell. Growth Differ. 1:47-51; Lai, E. et al. (1991) Trends Biochem. Sci. 16:427-30; DeSimone, V. et al. (1992) Biochem. Biophys. Acta 1132:119-126; and Crabtree, G. et al. (1992) in Transcriptional Regulation, S. S. McKnight and K. R. Yamamato (eds.) Cold Spring Harbor Laboratory, Cold Spring Harbor, N.Y., pp. 1063-1102).

Development of Pancreas and Pancreatic Stem Cells

During embryonic development, the pancreas derives from two separate outgrowths of dorsal and ventral foregut endoderm to form dorsal and ventral buds. These buds then fuse to form the definitive pancreas (Houssaint, E. (1980); Spooner, B. et al. (1970) J. Cell Biol. 47:235-46; Rutter, W. et al. (1980) Monogr. Pathol. 21:30-38; Guaidi, R. et al. (1996) Genes Dev. 10:1670-82; Zaret, K. (2000) Mech. Dev. 92:83-88; Edlund, H. (1998) Diabetes 47:1817-1823; St-Onge, L. et al. (1999) Curr. Opin. Gene Dev. 9:295-300; and Slack, J. (1995) 121:1569-80). During embryogenesis, islet development within the pancreas appears to be initiated from undifferentiated precursor cells associated primarily with the pancreatic ductal epithelium (Pictet, R. et al. (1992) in Handbook of Physiology, Steiner, D. and Frienkel, N. (eds.) Williams and Wilkins, Baltimore, Md., pp. 25-66). This ductal epithelium rapidly proliferates, and then subsequently differentiates into the various islet-associated cell populations (Teitelman, G. et al. (1993) Development 118:1031-39; and Beattie, G. et al. (1994) J. Clin. Endo. Met. 78:1232-1240). In the adult pancreas, the islet cell growth can occur through two different pathways: either by growth of new islets by differentiation of ductal epithelium (neogenesis), or by replication of preexisting β-cells. Neogenesis has been induced experimentally by dietary treatment with soybean trypsin inhibitors (Weaver, C. et al. (1985) Diabetologia 28:781-785), high level of interferon-γ (Gu, D. (1993) Dev. 118:33-46), partial pancreatectomy (Bonner-Weir, S. et al. (1993) Diabetes 42:1715-1720), wrapping of the head of the pancreas in cellophane (Rosenberg, L. et al. (1992) Adv. Exp. Med. Biol. 321:95-104), and by specific growth factors (Otonkonski, T. et al. (1994) Diabetes 43:947-952). Thus, it is generally accepted that all endocrine cell types of the pancreatic islets arise from the same ductal epithelial stem cell through sequential differentiation (Gu, D. (1993); Rosenberg, L. (1992); and Hellerstrom, D. (1984) Diabetologia 26:393-400). Pancreatic stem cells have been isolated from adult pancreatic ductal preparations, and have been shown to differentiate (to some degree) into insulin-producing cells in vitro (Ramiya, V. et al. (2000); Cornelius, J. et al. (1997) Horm. Metab. Res. 29:271-277; and Bonner-Weir, S. et al. (2000) PNAS 97(14):7999-8004), which upon transplantation, were able to reverse diabetes in non-obese diabetic (NOD) mice (Ramiya, V. et al. (2000)).

During embryonic development, there are differences in the specification of the dorsal and ventral pancreatic rudiments. The dorsal pre-pancreatic endoderm remains closely associated with the notochord during early developmental stages. Signals derived from overlaying notochord, such as activin and FGF-2, promote dorsal pancreas development by repressing endodermal expression of sonic hedgehog (Shh) (Hebrok M. et al. (2000) Dev. 127:4905-13; Kim, S. et al. (1997) Dev. 124:4243-52; and Li, H. et al. (1999) Nat. Genet. 23:67-70). Generation of a dorsal pancreas in response to these signaling events also requires the expression of a number of transcription factors. For example, mouse ‘knockout’ studies have shown that formation of the dorsal pancreas is dependent on Isl1 and Hlxb9, and that subsequent differentiation requires Pdx1 (Li, H. et al. (1999); Harrison, K. et al. (1999) Nat. Genet. 23:71-75; Ahlgren, U. et al. (1997) Nature 385:257-60). The mechanisms regulating the onset of ventral pancreas development are not fully defined. The control of ventral pancreatic development would differ from that of dorsal pancreas because the notochord does not extend as far as ventral endoderm, and by default, the ventral endoderm does not express Shh. Moreover, ventral pancreatic development is normal in Isl1−/− and Hlxb9−/− mice (Deutsch, G. et al. (2001) Development 128:871-881; and Duncan, S. (2001) Nature Genetics 27:355-356). Pdx1 is required at an earlier stage in pancreas development (Jonsson, J. et al. (1994) Nature 371:606-609; Ahlgren, U. et al. (1996) Development 122:1409-1416; Stoffers, D. et al. (1997) Nat. Genet. 15:106-110; and Offield, M. et al. (1996) Development 122:983-995). Mice and humans lacking Pdx1 are apancreatic (Jonsson, J. et al. (1994); Ahlgren, U. et al. (1996); Stoffers, D. et al. (1997); and Offield, M. et al. (1996)). However, there seems to be other genes that act upstream of Pdx1 expression for the initial commitment of the gut endoderm to a pancreatic fate. Accordingly, the evagination of the epithelium and the initial commitment of dorsal and ventral pancreatic buds still take place in Pdx1 mutant mice, and insulin- and glucagon-positive cells still differentiate (Ahlgren, U. et al. (1996); and Offield, M. et al. (1996)). Later, Pdx1 and Hlxb9 expression in the pancreas become restricted to the insulin-producing β-cells (Li, H. et al. (1999); Harrison, K. et al. (1999); and Jonsson, J. et al. (1994)). Pdx1 is required for maintaining the hormone-producing phenotype of the β-cell by regulating the expression of a variety of endocrine genes, including insulin, GLUT2, glucokinase, and prohormone convertases (PC) 1, 2, and 3 (Ahlgren, U. et al. (1998) Genes Dev. 12:1763-68; Hart, A. et al. (2000) Nature 408:864-68; and Baeza, N. et al. (2001) Diabetes 50, Sup. 1:S36). The Pdx1 gene activation may be regulated by HNF3,β (Zaret, K. (1996) Annu. Rev. Physiol. 58:231-251) and NeuroD/β2 (Sharma, T. et al. (1997) Mol. Cell Biol. 17:2598-2404). Several homeodomain and basic helix-loop-helix (bHLH) transcription factors like ngn3, Isl1, Nkx2.2, Nkx6.1, Pax4, Pax6, and NeuroD/β2, have been shown to play an important role in the control of pancreatic endocrine cell differentiation (Edlund, H. (1998); St-Onge, L. et al. (1999); Sander, M. et al. (1997) J. Mol. Med. 75:327-340; Madsen, O. et al. (1997) Horm. Metab. Res. 29(6):265-270; and Gradwohl, G. et al. (2000) PNAS 97(4):1607-11). Of these genes, ngn3 has been reported to be critical for the development of all four endocrine cell lineages of the pancreas (Gradwohl, G. et al. (2000)). Pax4 appear to selectively control the development of insulin-producing β-cells and somatostatin-producing δ-cells (Sosa-Pineda, B. et al. (1997) Nature 386:399-402). Nkx6.1 has a highly restricted β-cell expression in the adult rat (Madsen, 0. et al. (1997)). Disruption of Nkx6. 1 in mice leads to loss of β-cell precursors and blocks β-cell neogenesis (Sander, M. et al. (2000) Dev. 127(24):5533-5540). Thus, it is essential to screen for these factors following differentiation procedures to determine the extent of differentiation.

Various growth factors, hormones, vitamins and chemicals, such as hepatocyte growth factor (HGF), glucagon-like peptide-1 (GLP-1), exendin-4, activin-A, β-cellulin, dexamethasone, nicotinamide, and sodium butyrate, have been shown to be effective in β-cell differentiation in vitro. HGF (Mashima, H. et al. (1996) Endocrinol. 137:3969-76), GLP-1 (Zhou, J. et al. (1999) Diabetes 48:2358-2366), exendin-4 (Zhou, J. et al. (1999)), dexamethasone, β-cellulin and activin-A (Mashima, H. et al. (1996) J. Clin. Invest. 97(7):1647-54) differentiate acinar cells into insulin-secreting cells. GLP-1 increases levels of β-cell cAMP and insulin gene transcription and stimulates glucose-dependent insulin release (Grucker, D. et al. (1987) PNAS 84:3434-3438). Administration of GLP-1 for 10 days to neonatal diabetic rats following partial pancreatectomy stimulated expansion of β-cell mass via induction of islet proliferation and neogenesis (Xu, G. et al. (2000) Diabetes 48:2270-76). GLP-1 also increases Pdx1 gene expression and binding capacity (Buteau, J. et al. (1999) Diabetes 49:1156-1164). Exendin-4 is a potent structural analog of GLP-1, and has a longer circulating half-life. It binds to GLP-1 receptor on islets with similar affinity to GLP-1, but increases cAMP levels 3-fold higher than GLP-1 at equimolar concentrations, making it a more effective agent for use in chronic animal studies (Garcia-Ocana, A. et al. (2001) JCE & M 86:984-988). Dexamethasone and sodium butyrate might promote β-cell differentiation as evidenced by increased insulin/DNA contents in porcine pancreatic islet-like cell clusters (Korsgren, O. et al. (1993) Ups. J. Med. Sci. 98(1):39-52). In pancreas cell line, RIN-m5F, sodium butyrate increases 2-fold both hexokinase and glucokinase activities, as well as, the glucokinase gene expression. Nicotinamide is a poly (ADP-ribose) synthetase inhibitor known to differentiate and increase β-cell mass in cultured human fetal pancreatic cells and mouse IPSCs (Ramiya, V. et al. (2000); and Otonkoski, T. et al. (1993) J. Clin. Invest. 92:1459-66) and prevents the development of diabetes in drug induced diabetic animal models as well as in the NOD mice (Uchigata, Y. et al. (1983) Diabetes 32:316-18; and Yamada, K. et al. (1982) Diabetes 31:749-753).

Manipulation of Pancreatic Stem and Liver Stem Cell Differentiation

In embryonic development, the liver and ventral pancreas both originate from the same location in the ventral foregut (Houssaint, E. (1980); Rutter, W. (1980); Guaidi, R. et al. (1996); Zaret, K. (2000); Deutsch, G. et al. (2001); and Zaret, K. (1996)). Therefore, it is possible, from the developmental point of view, that epithelial cells in these two organs may share common stem cells. A new study shows that a bipotential cell population exists in the embryonic endoderm that gives rise to both the liver and the pancreas. The decision by these cells to adopt either a pancreatic or hepatic cell fate is determined by their proximity to the developing heart (Deutsch, G. et al. (2001)). The default developmental program of the ventral endoderm is to become ventral pancreas. Several lines of evidence have attested to the ability of pancreatic stem cells to differentiate into liver cell. For instance, copper depletion and repletion result in the atrophy of exocrine pancreas and the appearance of oval cells within the pancreatic ducts which then differentiate into hepatocytes within the pancreas (Rao, M. et al. (1986) Cell Differ. 18:109-117; Rao, M. et al. (1988) Biochem. Biophys. Res. Commun. 156:131-136; and Reddy, J. et al. (1991) Dig. Dis. Sci. 36(4):502-509). Oval cells with immunophenotype identical to hepatic stem cells were also found in human pancreas with acute pancreatitis, chronic pancreatitis, and pesidioblastosis (Mikami, Y. et al. (1998) Hepatology 28(4), Pt. 4:417A). The pancreatic hepatocytes respond to the carcinogens in a fashion similar to liver hepatocytes (Rao, M. et al. (1991) Am. J. Pathol. 139(5):1111-1117). Following transplantation into the liver, pancreatic oval cells isolated from copper-deficient rat pancreas can differentiate into mature hepatocytes with structural integration in the hepatic parenchyma and expression of biochemical functions unique to the hepatocytes (Dabeva, J. et al. (1997) PNAS 94:7356-61). Most recently, Wang and coworkers demonstrated the existence of undifferentiated progenitors of hepatocytes in the pancreas of normal adult mouse (Wang, X. et al. (2001) Am. J. Pathol. 158:571-79). Pancreatic cells can also be converted into hepatocytes in vitro by treatment with dexamethasone (Shen, C -N. et al. (2000) Nature Cell Biol. 2:879-887). The initial events involve activation of the transcriptional factor C/EBP-β. Transfection of cells with C/EBP-β brings about hepatic differentiation. Therefore, C/EBP-β is suggested as a key component that distinguishes the liver and pancreatic programs of differentiation. The consistent development of pancreatic hepatocytes in transgenic mice overexpressing KGF driven by insulin promoter indicates the involvement of KGF in the transdifferentiation process (Krakowski, M. et al. (1999) am. J. Pathol. 154(3):683-91). Although not specific to endocrine cells, there is also a report on the ability of liver to generate pancreatic epithelial cells (Rao, M. et al. (1986) Histochem. Cytochem. 34:197-201; and Bisgaard, H. et al. (1991) J. Cell Physiol. 147(2):333-343). Further, liver transduced with recombinant-adenovirus carrying gene encoding Pdx1 can produce functional insulin and ameliorates streptozotocin-induced diabetes in mice; however, Pdx1 is reported to not transdifferentiate liver hepatocytes to insulin producing cells in vitro, and no evidence is provided that mouse liver stem or progenitor cells are transfected in vivo (or in vitro) with the Pdx1 construct (Ferber, S. et al. (2000) Nature Med. 6(5):568-571). Finally, while sharing of transcription factors such as Isl1, ngn3, NeuroD/β2, Pax4, pax6, and Nkx2.2, between endocrine and neuronal differentiation pathways has been established (Ahlgren, U. et al. (1997); Sander, M. et al. (1997); Sosa-Pineda, B. et al. (1997); Pfaff, S. et al. (1996) Cell 84:309-320; Lee, J. et al. (1995) Science 268:836-844; Naya, F. et al. (1997) Genes Dev. 11:2323-2334; Miyata, T. et al. (1999) Genes Dev. 13:1647-52; St-Onge, L. et al. (1997) Nature 387:406-409; Ericson, J. et al. (1997) J. Cell 90:169-180; Sussel, L. et al. (1998) Dev. 125:2213-2221; Briscoe, J. et al. (1999) Nature 398:622-627), there is no clear information on the sharing of transcription factors between liver and pancreas.

### SUMMARY OF THE INVENTION

The subject invention comprises methods of culturing liver stem/progenitor cells with combinations of hormones, growth factors, vitamins and chemicals to convert the liver stem or progenitor cells to pancreatic functional cells. It further comprises transfection methods for conversion of liver stem or progenitor cells to pancreatic functional cells.

Thus, the invention provides a method for converting a liver stem/progenitor cell to the pancreatic functional cell by transfecting the liver stem/progenitor cell with a pancreatic development gene. Alternatively, the liver stem/progenitor cell may be cultured under conditions that convert the cell to the pancreatic functional cell. Further, conversion can be achieved by both transfection and culture conditions, effected simultaneously or sequentially in either order.

The liver stem/progenitor cell can be a hepatoblast or a liver oval cell. It is preferred that the liver stem/progenitor cell express at least one hematopoietic marker and/or at least one liver oval or hepatoblast cell marker. The hematopoietic markers include CD34, Thy1.1 and CD45. The liver hepatoblast or oval cell markers include α-fetal protein, albumin, cytokeratin 14 (CK14), c-kit, OC.2, OC.3, OC.10, OV1 and OV6.

The pancreatic development gene is any gene that is capable of converting liver stem/progenitor cells to pancreatic functional cells, and includes Pdx1, Hlxb9, Isl1, ngn3, Nkx2.2, Pax6, NeuroD/β2, Nkx6.1 and Pax4. Preferably, the pancreatic development gene is Pdx-1.

Culture conditions that convert liver stem/progenitor cells to the pancreatic functional cells comprise basal medium plus the added factors of hormones, growth factors, vitamins and chemicals or any combination thereof that induce differentiation into pancreatic cells. Such hormones include dexamethasone, glucagon-like peptide-1 (GLP-1), and exendin-4; growth factors include gastrin, interferon-γ (IFNγ), hepatocyte growth factor (HGF), epidermal growth factor (EGF), β-cellulin, activin-A, keratinocyte growth factor (KGF), fibroblast growth factor (FGF), transforming growth factor-α (TGF-α), transforming growth factor-β (TGF-β, nerve growth factor (NGF), insulin-like growth factors (IGFs), islet neogenesis associated protein (INGAP), and vascular endothelial growth factor (VEGF); vitamins include nicotinamide and retinoic acid; and chemicals include sodium butyrate.

According to this method, the liver stem/progenitor cell that is converted can express any combination of a number of pancreatic messenger RNAs, including insulin I (InsI), insulin II (InsII), glucagon, somatostatin, pancreatic polypeptide (PP), amylase, elastase, glucose transporter 2 (GLUT2), glucokinase, PC1, PC2, PC3, carboxypeptidase E (CPE), Pdx1, Hlxb9, Isl1, ngn3, Nkx2.2, Pax6, NeuroD/β2, Nkx6.1 and Pax4. Likewise, the converted cell may express any combination of a number of pancreatic proteins including InsI, InsI, glucagon, somatostatin, PP, amylase, elastase, GLUT2, glucokinase, PC1, PC2, PC3, CPE, Pdx1, Hlxb9, Isl1, ngn3, Nkx2.2, Pax6, NeuroD/β2, Nkx6.1 and Pax4.

Preferably, the converted liver stem/progenitor cell differentiates into the pancreatic endocrine pathway. Such converted cells can be cultured to produce endocrine hormones (e.g., insulin, glucagon and somatostatin from β, α and δ cells).

The method of conversion via transfection with a pancreatic development gene or via culture conditions may result in pancreatic cells at different stages of differentiation, including islet producing stem cells (IPSCs), islet progenitor cells (IPCs) and islet-like structures or IPC-derived islets (IdIs), or cellular components thereof (α, β, δ and/or PP cells). Transdifferentiation may also result in a cell that manifests expression patterns of a pancreatic cell (e.g., insulin production), and that may also retain characteristics of the liver stem/pancreatic cell (e.g., liver stem or progenitor markers). Liver stem/progenitor cell markers include hematopoietic markers and liver oval or hepatoblast cell markers.

All references cited herein are incorporated in their entirety by reference.

### BRIEF DESCRIPTION OF THE FIGURES

FIG. 1 sets forth the characterization of five liver epithelial cell lines derived from allyl alcohol-injured rat liver. The meaning of symbols is: − negative, +/− weakly positive, + positive, ++ strongly positive.

FIG. 2 illustrates the bipotentiality of liver epithelial line 3(8)#21 to differentiate into hepatocyte-like and bile duct-like cells. A: 3(8)#21 cultured without feeder are positive for mature hepatocyte marker H4. B: on day 6, 3(8)#21 cultured without feeder are positive for mature hepatocyte marker CYPIAII on day 12. C: 3(8)#21 cultured without feeder but with bFGF; more H4 positive cells observed on day 6. D: 3(8)#21 cells cultured on matrigel without feeder form ductular structure on day 4. E: 3(8)#21 cells cultured on matrigel without feeder express strongly mature bile duct cell marker BD1 on day 13. The magnification is 400×for panels A, B, C, and E. The magnification is 200×for panel D (Yin, L. et al. (2001A); Yin et al. (2001B); and Yin, L. et al. (2002)).

FIG. 3 shows the expression of pancreatic development markers in five liver stem/progenitor cell lines.

FIG. 4 illustrates the expression of insulin II and amylase in the liver progenitor lines after transfection with the Pdx1 gene.

### DETAILED DESCRIPTION

To facilitate a further understanding of the invention, the following definitions are provided.

“Islet producing stem cell” (IPSC) refers to those stem cells that arise from or among pancreatic ductal epithelium in vitro and in vivo. Methods for obtaining and maintaining IPSCs are described in detail in PCT/USOO/26469, filed Sep. 27, 2000, which incorporated herein in its entirety by reference.

“Islet progenitor cell” (IPC) refers to pancreatic progenitor cells that arise from IPSCs cultured in vitro using methods described herein and in PCT/US00/26469.

“IPC-derived islet” (IdI) refers to the islet-like structures that arise from IPCs cultured in vitro using methods described herein and in PCT/USOO/26469.

“Liver stem/progenitor cell” refers to all liver stem and/or progenitor cells, including without limitation, hepatoblasts, oval cells, liver epithelial cells with stem-like properties, and de-differentiated hepatocytes and bile duct cells. While many liver stem/progenitor lines have been reported in the literature (Williams, G. et al. (1971) Exp. Cell Res. 69:106-112; Williams, G et al. (1973) 29:293-303; Grisham, J. (1980) Ann. N.Y. Acad. Sci. 349:128-137; Tsao, M-S. et al. (1984) Exp. Cell Res. 154:38-52; Coleman, W. et al. (1997) Am. J. Pathol. 151:353-359; Coleman, W. et al. (1993) Am. J. Pathol. 142:1372-82; McCullough, K. et al. (1994) Cancer Res. 54:3668-71; Amicone, L. et al. (1997) EMBO J. 16:495-503; Spagnoli, F. et al. (1998) J. Cell Biol. 143:1101-1112; Sell, S. et al. (1982) Hepatol. 2:77-86; Shinozuka, H. et al. (1978) Cancer Res. 38:1092-98; McMahon, J. et al. (1986) Cancer Res. 46:4665-71; Brill, S. et al. (1999) Digest. Dis. Sci. 44:364-71; and Rogler, L. (1977) Am. J. Pathol. 150:591-602), preferably, the liver stem/progenitor cells used in the subject methods are obtained from liver injury models without the involvement of carcinogens, as described for example in Yin, L. et al. (2001A), Yin, L. et al. (2001B) and Yin, L. et al. (2002). It is also preferred that the liver stem/progenitor cells express one or more of the liver oval or hepatoblast cell markers (α-fetal protein, albumin, cytokeratin 14 (CK14), c-kit, OC.2, OC.3, OC.10, OV1 and OV6), and/or one or more of the hematopoietic stem markers (CD34, Thy1.1 and CD45).

“Pancreatic endocrine lineage” refers to commitment to development into pancreatic endocrine cells.

“Pancreatic lineage” refers to commitment to development into pancreatic cells including endocrine, exocrine and/or duct cells.

“Pancreatic functional cells” refers to cells of the pancreatic lineage or cells that have been transdifferentiated or converted according to methods described herein, and which express mRNA or proteins that are characteristic of and specific to a pancreatic cell (e.g., insulin), and which may also retain characteristics of the liver stem/pancreatic cell (i.e., liver stem or progenitor markers). The pancreatic functional cell preferably is a glucose-responsive, insulin producing cell. It preferably produces and secretes insulin protein in response to glucose stimulation. The response is preferably within the normal range of insulin response for the mammalian species of interest. Such normal ranges are known in the art or are readily determinable.

37 Transfection” refers to any method known in the art by which a fragment or construct of nucleic acid containing a coding sequence may be introduced into a target cell (here, a liver stem/progenitor cell) resulting in the expression of the coding sequence in the target cell. Included within the fragment or construct are the requisite promoter and regulatory sequences for expression in the target cell.

Thus, the subject invention comprises a method of converting a liver stem/progenitor cell to a pancreatic functional cell, by transfecting the liver stem/progenitor cell with a pancreatic development gene, and/or by culturing said liver stem/progenitor cell in a medium comprising factors that induce differentiation into the pancreatic functional cell. The resulting pancreatic functional cell can be a cell of the pancreatic endocrine lineage, or can be a cell having an expression pattern that is intermediate between the liver stem/progenitor cell and cells of pancreatic lineage. The term “cells of the pancreatic lineage” means islet producing stem cells (IPSCs), islet progenitor cells (IPCs), islet-like structures or IPC-derived islets (IdIs), or naturally derived pancreatic endocrine cells (e.g., α, β and/or δ cells, or duct cells). Additionally, cells having an intermediate expression pattern are those that produce and secrete insulin protein in response to glucose stimulation, and which may express a marker of the liver stem/progenitor cell.

The liver stem/progenitor cells can be hepatoblasts and/or liver oval cells. The liver stem/progenitor cell expresses at least one hematopoietic marker and/or at least one liver oval or hepatoblast cell marker. The hematopoietic markers are CD34, Thy1.1 and/or CD45. The hepatoblast or oval cell markers α-fetal protein, albumin, cytokeratin 14 (CK14), c-kit, OC.2, OC.3, OC.10, OV1 and/or OV6.

In the transfection embodiment, the pancreatic development gene can be Pdx1, Hlxb9, Isl1, ngn3, Nkx2.2, Pax6, NeuroD/β2, Nkx6.1 and/or Pax4. Preferably, the pancreatic development is Pdx-1.

In the culture transdifferentiation embodiment, liver stem/progenitor cells are cultured under methods known in the art in a standard medium plus factors. The factors include dexamethasone, glucagon-like peptide-1 (GLP-1), exendin-4, gastrin, interferon-γ (IFNγ), hepatocyte growth factor (HGF), epidermal growth factor (EGF), β-cellulin, activin-A, keratinocyte growth factor (KGF), fibroblast growth factor (FGF), transforming growth factor-α (TGF-α), transforming growth factor-β (TGF-β), nerve growth factor (NGF), insulin-like growth factors (IGFs), islet neogenesis associated protein (INGAP), vascular endothelial growth factor (VEGF), nicotinamide, retinoic acid, sodium butyrate or any combination thereof.

The converted cell can express any of a number of pancreatic messages including insulin I (InsI), insulin II (InsII), glucagon, somatostatin, pancreatic polypeptide (PP), amylase, elastase, glucose transporter 2 (GLUT2), glucokinase, PC1, PC2, PC3, carboxypeptidase E (CPE), Pdx1, Hlxb9, Isl1, ngn3, Nkx2.2, Pax6, NeuroD/β2, Nkx6.1 and/or Pax4. Accordingly, the converted cell can express pancreatic proteins including InsI, InsII, glucagon, somatostatin, PP, amylase, elastase, GLUT2, glucokinase, PC1, PC2, PC3, CPE, Pdx1, Hlxb9, Isl1, ngn3, Nkx2.2, Pax6, NeuroD/β2, Nkx6.1 and Pax4. It is preferred, however, that the converted cell produce and secrete insulin protein in response to glucose stimulation. The response is preferably within normal range for the mammalian cell of interest.

The subject invention also comprises a pancreatic functional cell produced by the methods described herein, wherein the pancreatic functional cell has an expression pattern that is intermediate between that of the liver stem/progenitor cell and cells of pancreatic lineage. In one embodiment, the pancreatic functional cell expresses Pdx1, amylase and insulin II.

The invention further comprises a method for producing an endocrine hormone comprising converting the liver stem/progenitor cells to pancreatic functional cells as described herein, culturing said pancreatic functional cells using methods known in the art and recovering endocrine hormone from the cell culture using methods known in the art.

### EXAMPLES

### Example 1

### Liver Stem/Progenitor Cells

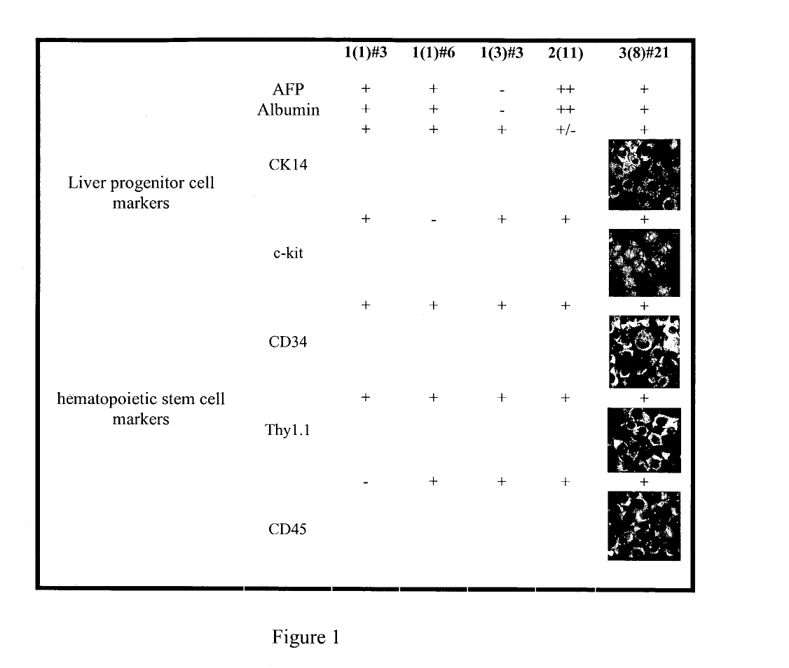

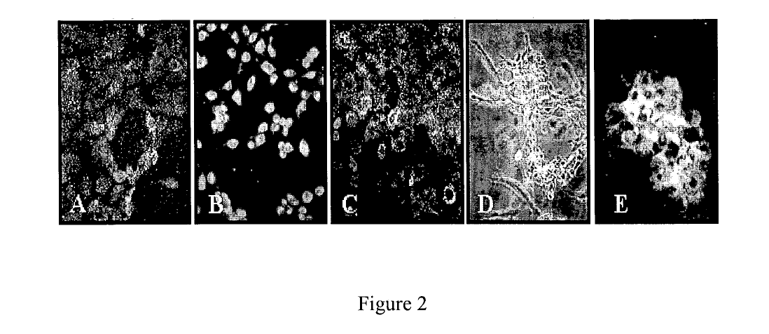

Liver epithelial cell lines with liver stem cell properties were developed from allyl alcohol (AA)-injured adult rat liver as described in Yin, L. et al. (2001 A); Yin, L. et al. (2001B); and Yin, L. et al. (2002). AA induces periportal liver injury, which is a liver injury model without the involvement of hepatocarcinogens (Peterson B. E. et al. (1998) Hepatology 27(4):1030-38). Five cell lines, named 1(1)#3, 1(1)#6, 1(3)#3, 2(11) and 2(8)#21, were chosen to investigate their potential of differentiation to pancreatic lineage cells. These 5 lines have been well characterized by Western blot, Northern blot, immunocytochemistry and histochemistry for various liver developmental, cell lineage markers and hematopoietic stem cell markers. The results are summarized in FIG. 1. Pictures of the immunocytochemistry results of the line 3(8)#21 are also presented in FIG. 1. Interestingly, almost all of the lines express hematopoietic stem cell markers CD34, Thy1.1, and CD45 indicating their possible relationship to hematopoietic stem cells. They also express liver progenitor cell genes such as ac-fetal protein (AFP), albumin, cytokeratin 14 (CK14) and c-kit. They do not express Ito cell marker Desmin or Kupffer cell/macrophage markers, ED1 and ED2 (results not shown). These cells can be maintained in their undifferentiated status without the expression of mature hepatocyte-specific genes such as glucose-6-phosphatase (G-6-Pase), dipeptidyl peptidase IV (DPPIV), and cytochrome P450 (CYP450), and without showing the expression of mature bile duct cell-specific gene CK19 (results not shown). All 5 lines are diploid by flow cytometry. Induction of differentiation carried out in line 3(8)#21 shows that hepatocyte phenotype can be induced by long-term culture without STO fibroblast feeder layer (FIG. 2A, B). Basic FGF is able to augment the differentiation (FIG. 2C). Culturing the cells on matrigel induces liver bile duct phenotype (FIG. 2D, E). These results suggest bipotentiality of line 3(8)#21 to differentiate into hepatocytes or liver biliary cells.

### Example 2

### Expression of Pancreatic Developmental and Cell Lineage Specific Genes in Liver Progenitor Cell Lines

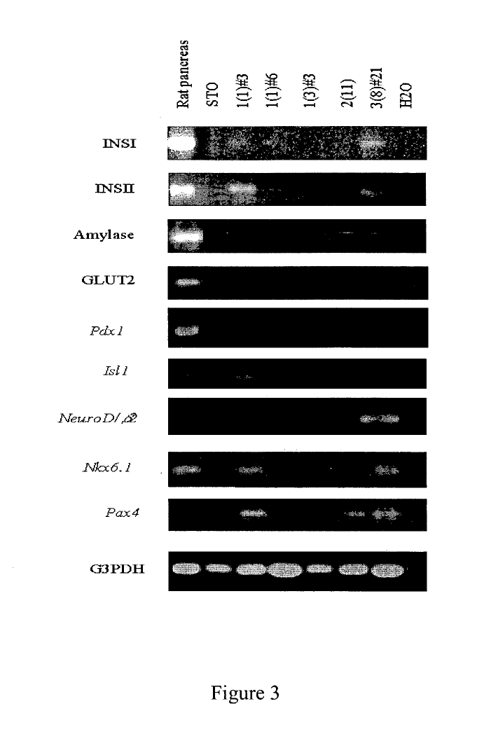

These five (untreated) liver progenitor cell lines have been analyzed for the expression of selected pancreatic endocrine markers including insulin I and insulin II, pancreatic exocrine marker amylase, GLUT2 (glucose transporter), and some of the transcription factors that are critically involved in the development of pancreas such as Pdx1, Isl1, NeuroD/β2, Nkx6.1 and Pax4. The expression of these genes was determined by RT-PCR, and confirmed by Southern blot. The data is presented in FIG. 3. Rat pancreatic tissue expresses most of the markers tested including insulin I, insulin II, amylase, GLUT2, Pdx1, Isl1, and Nkx6.1 but not NeuroD/β2 and Pax4 (FIG. 3; lane 1). Gamma-irradiated STO feeder cells do not express any of these markers (FIG. 3; lane 2). Liver progenitor cell lines 1(1)#3 (FIG. 3; lane 3) and 3(8)#21 (FIG. 3; lane 7) express almost all the pancreatic transcription factors tested and even insulin I and II, but they do not express detectable levels of amylase, Pdx1 and GLUT2 (FIG. 3; lanes 3 & 7). Cell line 1(1)#6 is positive for INSI and II and NeuroD/β2 (FIG. 3 lane 4). Cell line 2(11) are positive for NeuroD/β2, Nkx6.1 and Pax4 but negative for all other markers (FIG. 3; lane 6). Cell line 1(3)#3 is only positive for NeuroD/β2 (FIG. 3; lane 5). These data indicate that at least 2 of the 5 liver progenitor lines (1(1)#3 and 3(8)21) tested express their “preparedness” to enter into pancreatic pathway even before any treatment with islet-differentiating factors.

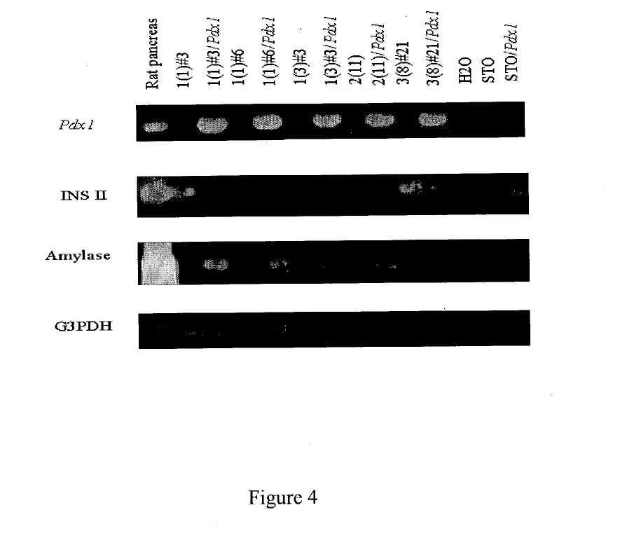

Additionally, and as a positive control, the pancreas-determining transcription factor, Pdx1 was transfected into each of these liver progenitor lines with the aim of directing the liver stem cells into pancreatic differentiation pathway. As shown in Figure 4, the introduction of Pdx1 gene triggers the expression of amylase gene (FIG. 4; lanes 3,5,7,9,11), which is not expressed in the non-transfected parental lines (FIG. 4; lanes 2,4,6,8,10). Interestingly, cell lines 1(1)#3, and 3(8)#21 which express insulin II exhibit reduction of insulin II expression following transfection with Pdx1 gene (FIG. 4; lanes 2,3,10,11) while induction of insulin II was seen in cell lines that did not express the gene prior to transfection (FIG. 4; lanes 4,5,6,7).

### Example 3

### Characterization of Expression in Liver Stem/Progenitor Cells under Different Experimental Conditions so as to Determine their Differentiation Potential

Liver progenitor lines described herein are studied for expression of genes controlling the pancreatic development at different stages (Pdx1, Hlxb9, Isl1, ngn3, Nkx2.2, Pax6, NeuroD/β2, Nkx6.1, and Pax4), endocrine cell lineage markers (insulin I, insulin II, glucagon, somatostatin, and PP), exocrine markers (amylase and elastase), and the genes associated with insulin sensing, synthesis, process and secretion (GLUT-2, glucokinase, PC1, PC2, PC3 and carboxypeptidase E (CPE)). Each cell line is evaluated for expression under untreated and treated conditions. Treated cell lines are those that are grown under culture conditions known to enhance differentiation of pancreatic stem or progenitor cells, and/or are transfected with pancreatic development genes.

RT-PCR, Southern blot, immunocytochemistry, and Western blot techniques are used to determine the gene expression both at mRNA expression (all genes) and at protein levels (e.g. Pdx1 and hormones). Normal pancreatic tissue, primary hepatocytes, and STO feeder cells serve as controls. The treated cell lines are characterized and compared to untreated lines. Expression of liver stem cell markers (AFP, albumin, CK14, c-kit, OV6, OV1) and hematopoietic stem/progenitor cell markers (CD34, Thy1.1, CD45) are also analyzed in the treated lines to see if their liver stem cell phenotypes are lost after treatment.

Plasmids such as plasmid pBKCMV/St1 (Pdx1) carrying Pdx1 gene and Neo gene (a gift from Dr. Dutta, Hoffmann-La Roche, Inc. Nutley, N.J.), are used to transfect liver cell lines. The Pdx1 transfected cells can be used as a positive control for differentiation into insulin-producing cells.

RT-PCR/Southern Blot

DNA-free RNA is extracted by using StrataPrep™ Total RNA Miniprep Kit (Stratagene, La Jolla, Calif.) or RNAqueous™-4PCR Kit (Ambion, Austin, Tex.) by following the manufacture's protocol. RT-PCR is carried out following methods known in the art. The oligonucleotides used as amplimers for PCR are listed in Table 1. PCR cycle is at 95° C. for 3 min followed by 94° C. for 45 sec, corresponding optimized annealing temperature for each primer pair is 45 sec, 72° C. for 1 min (34 cycles), and 72° C. for 10 min. PCR products are run in 1.5% Seakem agarose gel in TBE buffer using a BioRad/RAC300 power supply at 100 volt for 80 min. The gel is incubated in 1% ethidium bromide solution in TBE buffer for 15 to 30 min, and then viewed using UV light. Image is photographed and processed using AlphaImage™2200 Documentation & Analysis system (Alpha Innotech Corporation, San Leandro, Calif.). Digoxigenin-labeling of an Oligo probe for Southern blotting is carried out by using Dig Oligonucleotide Tailing Kit (Roche Molecular Biochemicals, Indianapolis, Ind.) by following the manufacture's protocol. As a corroborative technique, Southern blotting is carried out following the PCR reaction using the standard protocol.

TABLE 3
Oligonucleotides used as amplimers for PCR.

| | | | Size | GenBank accession |
|---|---|---|---|---|
| Gene | Sense primer | Antisense primer | (bp) | number |
| PdxI | ACATCTCCCCATACGAAGTGCC | GGAGCTGGCAGTGATGCTCAACT | 364 | U04833(R) |
| Isll | ACGTCTGATTTCCCTATGTGTTGG | CCGCTCTAAGGTGTACCACATCGA | 276 | S69329(R) |
| Hlxb9 | CAGCACCCGGCGCTCTCCTA | GAACTGGTGCTCCAGCTCCAGCAGC | 250 | NM-005515(Hu) |
| Ngn3 | CCTGCAGCTCAGCTGAACTTGGCGA | GCTCAGTGCCAACTCGCTCTT | 485 | AJ133776(Hu) |
| Nkx2.2 | CCGAGAAAGGTATGGAGGTGAC | CTGGGCGTTGTACTGCATGTGCTG | 187 | X81408(Ha) |
| Nkx6.1 | ATGGGAAGAGAAAACACACCAGAC | GAACGAGGAGGACGACGACGATTA | 280 | AF004431(R) |
| Pax4 | TGGCTTTCTGTCCTTCTGTGAGG | TCCAAGACTCCTGTGCGGTAGTAG | 214 | AF053100(R) |
| Pax6 | AAGAGTGGCGACTCCAGAAGTTG | CCTGAAGCAAGGATACAGGTGTGGT | 545 | U69644(R) |
| NeuroD/β2 | AGCCATGAATGCAGAGGAGGAC | ACACTCTGCAAAGGTTTGTCC | 400 | AF107728(R) |
| Insulin I | ATGGCCCTGTGGATGCGCTT | CTGGAGAACTACTGCAACT | 331 | J00747(R) |
| Insulin II | ATGGCCCTGTGGATCCGCTT | GTGACCTTCAGACCTTGGCA | 243 | V01243(R) |
| Glucagon | GTGGCTGGATTGTTTGTAATGCTG | GTTGATGAACACCAAGAGGAACCG | 236 | NM-012707(R) |
| PP | TGAACAGAGGGCTCAATACGAAAC | GATTTGTAGCCTCCCTTCTGTCT | 214 | M18207(R) |
| Amylase | GCCTTGGTGGGAAAGATATC | TCCCAAGGAAGCAGACCTTT | 510 | V01225(R) |
| Elastase | GTGAGCAGCCAGATGACTTTCC | CCTGGATGAACAATGTCATTG | 573 | NM-012552(R) |
| GLUT-2 | TTAGCAACTGGGTCTGCAAT | CATGAGTGTAGGACTACACC | 343 | J03145(R) |
| Glucokinase | AGAGTGATGCTGGTCAAAGTGGGA | ATGATTGTGGGCACTGGCTGCAAT | 440 | J04218(R) |
| G3PDH | GCCATCACTGCCACCCAGAAG | GTCCACCACCCTGTTGCTGCA | 440 | M32599(R) |

Ha, hamster; Hu, human; R, rat.

Immunocytochemistry

An avidin-biotin method adapted from Biogenex (San Ramon, Calif.) is followed. Cells are either cytospun onto Fisher Brand superfrost plus slides (Fisher Scientific, Pittsburgh, Pa.) using a cytocentrifuge (Cytopro M, Wescor, Inc. Logan, Utah) or grown onto 8 well glass slides (ICN Costa Mesa, Calif.) placed in tissue culture plates (Falcong, Becton Dickinson, Franklin Lakes, N.J.), and fixed in 0.5% glutaraldehyde for 1 hr at room temperature. For intracellular staining, the cells are permeabilized using 0.2% Triton-X. Blocking and antigen retrieval (when necessary) is done prior to primary antibody staining of the cells. Secondary antibodies are conjugated to biotin which is linked to alkaline phosphatase or horseradish peroxidase; and streptavidin, which binds to the biotin, is linked to alkaline phosphatase or peroxidase. Antibodies are then visualized using 3,3′-diaminobenzidine (DAB), 3-amino-9-ethylcarbzole (AEC), Fast Red, or 5-bromo-4-chloro-3-indolyl phosphate/nitro blue tetrazolium (BCIP/NBT). This system also can be used for double staining which can visualize multi-antigen expression.

Western Blot

Western blot is also used for the gene translation study. Cells are lysed with lysis buffer (for 8.0 ml: 3.8 ml of dH₂O, 1 ml of 0.5 M Tris-HCl (pH 6.8), 0.8 ml of glycerol, 1.6 ml of 10% (w/v) SDS, and 0.4 ml of -mercaptoethanol, and 0.4 ml of 0.5%

TABLE 4
Factors and their final concentrations in the medium

Basal Medium (BM) Components
DMEM (Dulbecco's Minimum Essential Medium, Gibco-BRL)
0.1 mM 2-Mercaptoethanol
15% FBS (HyClone)
1x ITS (insulin, transferrin and selenium) (Gibco-BRL)
Penicillin 100 IU/ml and streptomycin 100 μg/ml
Fungizone ® 1 μg/ml (Gibco-BRL)
200 mM L-glutamine
1X non-essential amino acids (Gibco-BRL)
Factors to be added to BM
Dexamethasone 10⁻⁷ M
GLP-1 (glucagon-like peptide) 10 nM
Exendin-4 (10 nM)
Gastrin (10 nM)
IFNγ (interferon-γ) 0.1-2 ng/ml
HGF (hepatocyte growth factor) 20 ng/ml
EGF (epidermal growth factor) 10 ng/ml
Betacellulin 5 nM
Activin A 1 nM
KGF (keratinocyte growth factor) 5-10 ng/ml
FGF (fibroblast growth factor) 10 ng/ml
TGF-alpha (transforming growth factor) 10-15 ng/ml
TGF-beta
NGF (nerve growth factor) 25-50 ng/ml
IGFs (insulin-like growth factor) 10 ng/ml
INGAP (islet neogenesis associated protein) 125 ng/ml
VEGF (vascular endothelial growth factor) 10-20 ng/ml
Nicotinamide 10 mM
Retinoic Acid 1 ng/ml
Sodium butyrate 2.5 mM

bromophenol blue). Tissues are homogenized on ice in homogenization buffer (20 mM Tris, 137 mM NaCl, 10% glycerol, 1 mM Na₃VO₄, 1 u/ml aprotinin, 1 mM 4-(2-aminoethyl) benzenesulfonyl fluoride (AEBSF), pH 8.0), centrifuged at 9,000 g for 20 min at 4° C., and the supernatant collected. The protein concentration is determined using Coomassie Plus Protein Assay Reagent (PIERCE, Rockford, Ill.). Samples are then run on separating gel at appropriate concentration at 100 volts, 4 watts, and 50 mAs for 2 hr. Gel is then transferred using Mini Trans-Blot Cell (Bio-Rad, Hercules, Calif.) to nitrocellulose membrane (Bio-Rad) in transfer buffer (25 mM Tris, 192 mM glycine and 20% v/v methanol, pH 8.3) at 30 volts, 2 watts, and 50 mAs overnight. The membrane is then blotted in respective primary antibody at 4° C. overnight. Thereafter, the membrane is washed and incubated with the corresponding secondary antibody linked with alkaline phosphatase for 1 hr at room temperature, and developed in carbonate buffer (0.1 M NaHCO₃, 1 mM MgCl₂, pH 9.8) containing 60 μl of nitro blue tetrazolium (NBT) solution (dissolve 50 mg of NBT in 0.7 ml of N,N-Dimethylformamide (DMF) with 0.3 ml dH₂O), and 60 μl of 5-bromo-4-chloro-3-indolyl phosphate (BCIP) solution (dissolve 50 mg of BCIP in 1 ml of 100% DMF) until appropriate color obtained.

Differentiation of Liver Stem Cells

Liver stem cells are cultured in basal medium (BM) containing combinations of hormones (dexamethasone, GLP-1, exendin-4), growth factors (gastrin, interferon-γ (IGFγ), HGF, EGF, β-cellulin, activin-A, KGF, FGF, TGF-α & -β, NGF, IGFs, INGAP, and VEGF), vitamins (nicotinamide, and retinoic acid), and/or chemicals (sodium butyrate), at concentrations listed in Table 4. The concentrations set forth in Table 4 may be varied by 1-3 orders of magnitude so as to optimize their effectiveness. The foregoing hormones, growth factors, vitamins and chemicals are reported in the literature or in PCT Application No. PCT/US02/0988 1, filed Mar. 29, 2002, to be involved in pancreas/β-cell development. Expression of liver stem cells markers and hematopoietic stem cell markers are also observed in the treated lines to determine whether their liver stem cell phenotypes are lost after treatment.

Transfection and Selection

FuGENE 6 transfection reagent (Roche Molecular Biochemicals, Indianapolis, Ind.) is used to transfect pBKCMV/Stf1 (Pdx1) carrying Pdx1 gene and Neogene into liver stem cell lines using manufacturer's protocol. Mock transfection and vector alone transfection are also done at the same time. Three days after gene transfection, the cells are cultured in the culture medium containing G418 1 mg/ml. The resistant clones are grown out in about ten days. The selection is carried out for 2 to 4 weeks. Thereafter, the cells are cultured in the culture medium containing 0.3 mg/ml of G418.

Analogous transfections can be carried out with plasmids containing other pancreatic development genes.

### Example 4

### Determination of the Functional Capability of Rat Liver Stem/Progenitor Cell-Derived Insulin-Producing Cells (LSDIPCs)

The cell lines of Example 3 that are found to produce insulin (LSDIPCs) are further evaluated for their glucose responsiveness. The cells are tested for both extra- and intracellular insulin production. Where a cell line demonstrates glucose responsive insulin production, then the substrate phosphorylation pattern can be determined following glucose stimulation. Freshly isolated rat islet cells serve as a positive control. Observation of substrate phosphorylation pattern reveals the early signaling events involved in the induction of insulin production.

Glucose Induced Insulin-Stimulation Assay

Differentiated LSDIPCs are seeded at a concentration of 2×10⁵ cells per well in 24 well plates with 1 ml of medium containing 5.5 mM glucose for 24 hr to rest. Cells are washed with Krebs-Ringer buffer (KRB) and are stimulated with 1 ml of culture medium with or without glucose (0, 5.5, 11 and 17.5 mM glucose) for 3-18 hrs. The cell free supernatant is collected and stored at −70° C. until use. The cells are then treated with lysis buffer to determine insulin content using Mercodia Ultrasensitive Rat Insulin ELISA Enzyme immunoassay kit (Mercodia, Uppsala, Sweden). This insulin kit is used to measure both secreted and intracellular insulin using BioRad's Benchmark plate reader (490 nm). The insulin values are normalized to total DNA concentrations (extracted using Triaol™, Gibco) of cells.

ELISAs for Hormone Detection

As mentioned above, secreted and intracellular insulin are measured using Mercodia Ultrasensitive Rat Insulin ELISA Enzyme immunoassay kit (Mercodia, Uppsala, Sweden) following the manufacturer's protocol. Similarly, a glucagon assay is carried out using methods known in the art or adaptations thereof.

Substrate Phosphorylation Assay

Differentiated LSDIPCs are homogenized in extraction buffer (20 mmol/l K₂HPO4, pH 7.5, 5 mmol/l DTT, 1 mmol/l EDTA, and 110 mmol/l KCL) following stimulation with 17.5 mM glucose for 0, 5, 15 and 30 min. The homogenate is used to separate protein on 10% SDS-PAGE (BioRad) and the phosphorylated protein substrates are detected using anti-phosphotyrosine antibody (Pharmingen, San Diego, Calif.) in Western blot technique.

It should be understood that the examples and embodiments described herein are for illustrative purposes only and that various modifications or changes in light thereof will be suggested to persons skilled in the art and are to be included within the spirit and purview of this application and the scope of the appended claims.

1. A method of converting a liver stem/progenitor cell to a pancreatic functional cell, wherein said pancreatic functional cell produces and secretes insulin in response to glucose stimulation, said method comprising:
transfecting said liver stem/progenitor cell with a pancreatic development gene, culturing said liver stem/progenitor cell in a medium comprising factors that induce differentiation into the pancreatic functional cell, or both, whereby said transfected cell is converted to the pancreatic functional cell.

2. The method of claim 1, wherein the pancreatic functional cell is a cell of the pancreatic endocrine lineage.

3. The method of claim 2, wherein said converted cell is selected from the group consisting of islet producing stem cells (IPSCs), islet progenitor cells (IPCs) and islet-like structures or IPC-derived islets (IdIs).

4. The method of claim 1, wherein the pancreatic functional cell further expresses a marker of the liver stem/progenitor cell.

5. The method of claim 1, wherein said liver stem/progenitor cell is selected from the group consisting of hepatoblasts and liver oval cells.

6. The method of claim 1, wherein said liver stem/progenitor cell expresses at least one marker selected from the group consisting of hematopoietic markers and liver oval or hepatoblast cell markers.

7. The method of claim 6, wherein said hematopoietic markers are selected from the group consisting of CD34, Thy1.1 and CD45.

8. The method of claim 6, wherein said liver hepatoblast or oval cell markers are selected from the group consisting of a-fetal protein, albumin, cytokeratin 14 (CK14), c-kit, OC.2, OC.3, OC.10, OV1 and OV6.

9. The method of claim 1, wherein said pancreatic development gene is selected from the group consisting of Pdx1, Hlxb9, Isl1, ngn3, Nkx2.2, Pax6, NeuroD/β2, Nkx6.1 and Pax4.

10. The method of claim 9, wherein said pancreatic development gene is Pdx-1.

11. The method of claim 1, wherein said factors are selected from the group consisting of dexamethasone, glucagon-like peptide-1 (GLP-1), exendin-4, gastrin, interferon-γ (IFNγ), hepatocyte growth factor (HGF), epidermal growth factor (EGF), β-cellulin, activin-A, keratinocyte growth factor (KGF), fibroblast growth factor (FGF), transforming growth factor-α (TGF-α), transforming growth factor-β (TGF-β), nerve growth factor (NGF), insulin-like growth factors (IGFs), islet neogenesis associated protein (INGAP), vascular endothelial growth factor (VEGF), nicotinamide, retinoic acid, sodium butyrate and any combination thereof.

12. The method of claim 1, wherein said converted cell expresses a pancreatic message selected from the group consisting of insulin I (InsI), insulin II (InsII), glucagon, somatostatin, pancreatic polypeptide (PP), amylase, elastase, glucose transporter 2 (GLUT2), glucokinase, PC1, PC2, PC3, carboxypeptidase E (CPE), Pdx1 , Hlxb9, Isl1, ngn3, Nkx2.2, Pax6, NeuroD/β2, Nkx6.1 and Pax4.

13. The method of claim 1, wherein said converted cell expresses a pancreatic protein selected from the group consisting of InsI, InsII, glucagon, somatostatin, PP, amylase, elastase, GLUT2, glucokinase, PC1, PC2, PC3, CPE, Pdx1 , Hlxb9, Isl1, ngn3, Nkx2.2, Pax6, NeuroD/β2, Nkx6.1 and Pax4.

14. A pancreatic functional cell produced by the method of claim 1.

15. The pancreatic functional cell of claim 14, wherein the cell further expresses Pdx 1, amylase and insulin II.

16. A method for producing an endocrine hormone comprising converting liver stem/progenitor cells according to the method of claim 1, and further comprising:
culturing said converted cells; and
recovering endocrine hormone from said cell culture.

17. A liver stem/progenitor cell that has been transfected with a pancreatic development gene.

18. The method of claim 17, wherein said liver stem/progenitor cell is selected from the group consisting of hepatoblasts and liver oval cells.

19. The liver stem/progenitor cell of claim 17 wherein the pancreatic development gene is selected from the group consisting of Pdx1, Hlxb9, Isl1, ngn3, Nkx2.2, Pax6, NeuroD/β2, Nkx6.1 and Pax4.

20. The liver stem/progenitor cell of claim 19 wherein the pancreatic development gene is Pdx1.

21. A liver stem/progenitor cell that has been cultured in a medium comprising factors that induce differentiation into a pancreatic functional cell.

22. The liver stem/progenitor cell of claim 21 wherein said factors are selected from the group consisting of dexamethasone, glucagon-like peptide-1 (GLP-1), exendin-4, gastrin, interferon-γ (IFNγ), hepatocyte growth factor (HGF), epidermal growth factor (EGF), β-cellulin, activin-A, keratinocyte growth factor (KGF), fibroblast growth factor (FGF), transforming growth factor-α (TGF-α), transforming growth factor-β (TGF-β), nerve growth factor (NGF), insulin-like growth factors (IGFs), islet neogenesis associated protein (INGAP), vascular endothelial growth factor (VEGF), nicotinamide, retinoic acid, sodium butyrate and any combination thereof.
